(19) United States
(12) Patent Application Publication
(10) Pub. No.: US 2018/0085034 A1
(43) Pub. Date: Mar. 29, 2018

(54) PREPULSE INHIBITION OF THE ACOUSTIC STARTLE REFLEX

(71) Applicant: Northeast Ohio Medical University, Rootstown, OH (US)

(72) Inventors: Alexander Galazyuk, Kent, OH (US); Ryan Longenecker, Rootstown, OH (US)

(21) Appl. No.: 15/678,543

(22) Filed: Aug. 16, 2017

Related U.S. Application Data
(60) Provisional application No. 62/375,557, filed on Aug. 16, 2016.

(51) Int. Cl. A61B 5/12; A61K 49/00; A61B 5/00

(52) U.S. Cl. CPC A61B 5/125; A61K 49/0004; A61B 2503/42; A61B 2503/04; A61B 2503/40; A61B 5/4848

(57) ABSTRACT

A method of evaluating hearing in a subject is described. The method includes delivering a pre-pulse auditory stimulus to the subject, allowing a stimulus gap from 10 to 200 milliseconds to pass, delivering an auditory startle stimulus to the subject, measuring the startle response of the subject, and determining if the startle response of the subject to the auditory startle stimulus has decreased compared with a control value for the startle response, repeating these steps until the pre-pulse auditory stimulus administered has a value sufficient to cause a decrease in the startle response of the subject, and determining that the subject has hearing loss if the pre-pulse auditory stimulus required to decrease the startle response is higher than a pre-pulse auditory stimulus threshold value for the subject. The method can be used to evaluate the effect of drugs on hearing, both in terms of hearing loss and improved hearing.

### CONTINUING APPLICATION DATA

This application claims the benefit of U.S. Provisional Application Ser. No. 62/375,557, filed Aug. 16, 2016, the disclosure of which is incorporated by reference herein.

### GOVERNMENT FUNDING

The present invention was made with Government support under Grant Nos. R01 DC011330 and 1F31 DC013498-01A1 awarded by the National Institute on Deafness and Other Communication Disorders. The Government has certain rights in the invention.

### BACKGROUND

Quick and accurate assessment of auditory thresholds is a prerequisite for experimental manipulations in the field of auditory research. To date, a variety of protocols have been used to determine audiometric thresholds. Auditory brainstem responses, behavioral audiograms, and startle reflex audiometry have each been used to assess hearing, yet each has limitations which should be taken into account when interpreting data related to threshold shifts and overall cochlear damage following sound or chemical lesions.

Perhaps the most ubiquitous test used to assess hearing performance is the auditory brainstem response. Rapid assessment of auditory brainstem circuitry makes ABR a good candidate for detecting gross changes in the auditory system. It is known that following an auditory insult, ABRs reliably identify elevated thresholds. This temporary threshold shift is thought to result from swelling of cochlear nerve terminals which is present for days after exposure. When measured with ABRs, thresholds return to baseline soon after noise exposure. Robertson D, Hearing Research 9, 263-278 (1983). However, recent work has clearly demonstrated that this measure does not account for the trauma-induced damage to ribbon synapses. Kujawa S G, Liberman M C, J Neurosci 29, 14077-14085 (2009). ABR wave one amplitudes have been shown to more accurately represent suprathreshold hearing loss, as they correlate strongly with ribbon synapse denervation following sound exposure. Liberman et al., J Neurosci 34(13): 4599-4607 (2014). However, the ABR methodology is not without some caveats. Immediately following sound exposure, ABR thresholds are often elevated so strongly that they cannot be accurately measured which also precludes wave one amplitudes from being assessed. Furthermore, ABR's are typically collected under anesthesia and are challenging to measure in animals with larger body mass. Lastly, some have contended that while ABRs thresholds are useful in detecting noise-induced damage to the auditory brainstem, they do not provide any perceptual indications of hearing loss. Alternatively, others have stated that ABRs can closely approximate behavioral thresholds in humans, yet ABRs are known to be less precise. For these reasons, it is useful to explore alternatives for hearing assessment.

Many years ago it was found that prepulse modulation of the acoustic startle reflex (ASR) could be employed to assess behavioral response thresholds. Fechter et al., J. Acoustic. Soc. Am. 84(1), 179-185 (1988). Prepulse inhibition, a decrease of ASR magnitude when a preceding weaker sound (prepulse) is presented before the startle, has been used for over half a century to objectively measure complex neurological systems. Hoffman H S, Searle J L, J Comp Physiol Psychol., 60(1), 53-581965 (1965); Gerrard R L, Ison J R, J Experimental Psych: Animal Behavior Processes 16(1): 106-112 (1990). Much like behavioral audiograms collected by operant conditioning methods (Radizwon et al., J. Comp. Physiol. A. Neuroethol. Sens. Neural Behay. Physiol., 195(10), 961-969 (2009)), the prepulse was varied in intensity and frequency to differentially modulate the startle response. This method has successfully identified behavioral correlates of cochlear damage due to ototoxic drugs (Young J S, Fechter L D, J. Acoust. Soc. Am. 73(5), 1686-1693 (1983)) and temporary threshold shifts due to pure tone acoustic exposure (Walter et al., Open Journal of Acousics 2, 34-49 (2012)).

### SUMMARY OF THE INVENTION

The inventors have developed a new method of hearing assessment utilizing prepulse inhibition (PPI) of the acoustic startle reflex, a commonly used tool that has been used in many fields of science. The inventors describe using PPI with some modifications to measures detection thresholds in multiple awake animals simultaneously. They found that in control mice PPI audiometric functions are similar to both ABR and traditional operant conditioning audiograms. The hearing thresholds assessed with PPI audiometry in sound exposed mice were also similar to those detected by ABR thresholds one day after exposure. However, three months after exposure PPI threshold shifts were still evident at and near the frequency of exposure whereas ABR thresholds recovered to the pre-exposed level. In contrast, PPI audiometry and ABR wave one amplitudes detected similar losses. PPI audiometry provides a high throughput automated behavioral screening tool of hearing in awake animals. Overall, PPI audiometry and ABR assessments of the auditory system are robust techniques with distinct advantages and limitations, which when combined, can provide ample information about the functionality of the auditory system.

Advantages of this approach for assessing hearing thresholds are numerous. First, the measure is based on a reflex and does not require experience in animal behavior and months of animal training as in other commonly used behavioral paradigms. Second, in contrast to the ABR approach, PPI audiometric functions can be collected in awake animals, avoiding confounds of anesthesia. Finally, a key advantage is that many animals can be tested at once with a short preparation, allowing for high data throughput, and timely data collections at various experimental conditions.

### BRIEF DESCRIPTION OF THE FIGURES

The present invention may be more readily understood by reference to the following figures, wherein:

FIG. 1 provides a flowchart showing a method of detecting hearing loss in a subject.

FIG. 2 provides a flowchart showing a method of determining a pre-pulse auditory stimulus threshold value for a subject

FIG. 3 provides a flowchart showing a method of determining the effect of a drug on a subject's hearing.

FIGS. 4A-4D provide graphs for determining detection thresholds for PPI audiometry. A. Schematic of stimulus presented, as described in the Example. B. Detection function. For each frequency, a cubic spline was fitted to medians of the square-root-transformed response amplitudes for SO and each intensity prepulse. Detection threshold was defined as the sound level where this function intersected the 95% lower confidence interval of a bootstrapped Startle Only response distribution (C). C. Calculations of Startle Only 95% confidence intervals. A Startle Only response amplitude distribution was created by bootstrapping square-root-transformed response amplitudes. The lower 95% confidence interval was used for detection threshold identification (in B). D. Threshold detection data exemplar. Representative audiometric functions (tested at 12.5 kHz) from an individual mouse before and after exposure. In the control condition the PPI function is both steep and monotonic with the detection threshold located at 9.4 dB SPL. In the exposed condition PPI functions tend to become shallower and non-monotonic, leading to a higher detection threshold at 64.3 dB SPL.

FIG. 5 provides a graph showing short inter-trial-intervals (ITIs) are effective in PPI sessions. PPI functions across frequencies (4-31.5 kHz) in six mice tested with short (4-6 s) and long (15-25 s) ITIs. Error bars show SEMs.

FIGS. 6A-6F provide graphs showing PPI audiometric functions are consistent across time and subjects. PPI audiometric functions from six mice (A-F) over 3 days of testing. Average thresholds were different based on the particular mouse and frequency tested. However, the variation between all conditions was only 5.77 dB (SEM), which suggests low threshold variability.

FIGS. 7A-7I provide graphs comparing PPI and ABR audiometric thresholds in the assessment of sound exposure. Thresholds from a group of 10 mice were recorded before (control), 1 day after, and 3 months after exposure. The narrow band exposure stimulus is represented by a grey box. Error bars show SEMs. A: Group PPI threshold averages as a function of frequency (4-31.5 kHz). B-C: PPI thresholds for two individual mice. D: Group ABR threshold averages as a function of frequency (4-31.5 kHz). E-F: ABR thresholds for two individual mice. G-I: Correlations between PPI and ABR thresholds from 10 mice in each condition. *=significant difference between the control condition and one day after exposure; #=significant difference between the control condition and three months after exposure. *=significant difference between the control condition and one day after exposure; #=significant difference between the control condition and three months after exposure; @=significant difference between one day after exposure and three months after exposure.

FIGS. 8A-8H provide graphs comparing prepulse modulated startle responses and resulting inhibition to ABR wave one amplitudes in the assessment of sound exposure. Measures from a group of 10 mice were recorded before (control), 1 day after, and 3 months after exposure. Startle (A1-F1): Prepulse modulated startle response magnitudes as a function of prepulse intensity, with each panel representing data from one pure tone frequency (4 kHz A1-31.5 kHz F1). PPI (A2-F2): Resulting inhibition curves from the prepulse modulated startle responses in Startle. Inhibition was calculated as Startle Only (SO) divided by prepulse+startle for each stimulus frequency/intensity combination. ABR (A3-F3): ABR wave one amplitudes (peak to peak) as a function of stimulus level, with each panel representing data from one pure tone frequency (4 kHz A2-31.5 kHz F2). G-H: Correlations between PPI and ABR amplitudes from 10 mice in each condition. *=significant difference between the control condition and one day after exposure; #=significant difference between the control condition and three months after exposure; @=significant difference between one day after exposure and three months after exposure.

FIGS. 9A and 9B provide graphs showing the frequency specific deficits in PPI and ABR wave one amplitudes after sound exposure. A: Change of PPI after exposure (values from 40 and 80 dB SPL prepulse levels FIG. 8A2-F2). B: Change of ABR wave one amplitudes (values from 40 and 80 dB SPL stimulus levels FIG. 5A3-F3). The narrow band exposure stimulus is represented by a grey box. The dashed line represents no change from control condition. Error bars show SEMs. *=significant difference between the control condition and one day after exposure; #=significant difference between the control condition and three months after exposure.

FIGS. 10A-10C provide graphs showing the effect of sound exposure on startle response magnitudes. Startle responses across a range of startle intensities were measured before (control), 1 day after, and 3 months after exposure. A: Group input/output function averages of 10 mice. B-C: input/output functions from two individual mice. Error bars show SEMs. *=significant difference between the control condition and one day after exposure; #=significant difference between the control condition and three months after exposure.

FIGS. 11A-11E provide graphs showing the relationship between startle response amplitudes and startle probability. A: Representative input/output startle-only function from one control mouse comparing startle response amplitudes (left Y axis) and corresponding startle response probabilities (right Y axis) as a function of startle stimulus intensity. B: Representative prepulse modulation function (20 kHz pure tone prepulse) from one control mouse comparing startle response amplitudes (left Y axis) and corresponding startle response probabilities (right Y axis) as a function of prepulse stimulus intensity. C-E: Correlations between startle response amplitudes and startle response probability for each condition measured with PPI audiometry (data from FIG. 9A1-F1). Note: x-axis scales are different to accommodate data because of net losses in startle response magnitudes after sound exposure. *=significant correlation.

FIG. 12 provides an image of a model for auditory nerve fiber damage three months after sound exposure. Post exposure, high threshold fibers across the range of sound exposure are damaged. Low threshold fibers are mostly spared except for a ⅓-octave notch above the center frequency of exposure (16 kHz region). This damage explains both the PPI audiometric thresholds seen in FIG. 7A-C, as well as the PPI and ABR wave one amplitude curves in FIGS. 8A2-F2 and FIGS. 8A3-F3 respectively.

### DETAILED DESCRIPTION OF THE INVENTION

The terminology as set forth herein is for description of the embodiments only and should not be construed as limiting of the invention as a whole. As used in the description of the invention and the appended claims, the singular forms “a”, “an”, and “the” are inclusive of their plural forms, unless contraindicated by the context surrounding such.

Methods of evaluating hearing using pre-pulse inhibition are described. Pre-pulse inhibition is an auditory effect in which an initial pulse of sound can inhibit the startle response that occurs in response to a subsequent loud noise (i.e., auditory startle stimulus). Because the methods rely on evaluation of the startle response, there is no subjective or conscious element to the test, which increases its reliability and ability to be used in subjects that are unable to provide conscious feedback on their response to auditory stimulus.

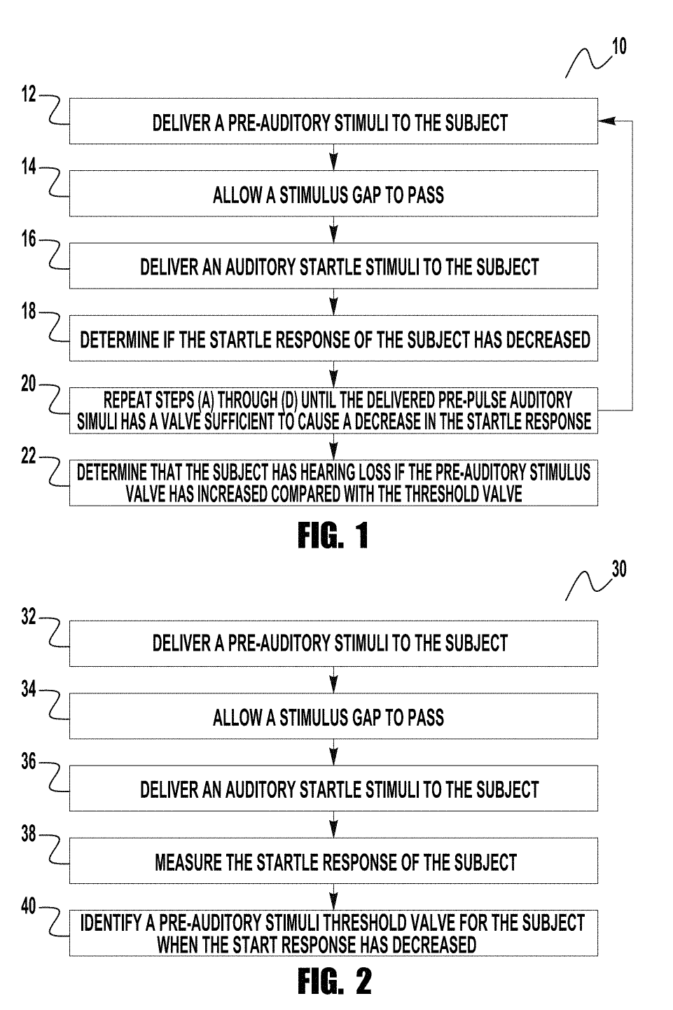

In one aspect, the present invention provides a method of detecting hearing loss in a subject 10, as shown in FIG. 1. The method includes the steps of delivering a pre-pulse auditory stimulus to the subject 12, allowing a stimulus gap to pass 14, delivering an auditory startle stimulus to the subject 16, and determining if the startle response of the subject has decreased 18. If the startle response of the subject does not show a decrease, these steps are repeated until the delivered pre-pulse auditory stimulus has a value sufficient to cause a decrease in the startle response of the subject 20. It can then be determined that the subject has hearing loss if the pre-pulse auditory stimulus required to decrease the startle response is higher than a pre-pulse auditory stimulus threshold value for the subject 22.

The method includes delivering auditory stimuli to the subject. Auditory stimuli are sound waves capable of being heard by a normal subject. The auditory stimulus can be generated and delivered by an audiometric unit. This can be a simple sound generator by means of which the user is exposed to test signals of different frequencies and sound levels through insert earphones, headphones, free speakers, or bone conduction transducers. The audiometric unit typically includes computer processor programmed/configured to run one or more audiometric test protocols, generating appropriate signals to cause the sound generator to generate one or more sound stimulus in one or both of the subject's ears in accordance with the programmed test protocols. The audiometric test regimen could be a full test designed to generate a complete audiometric profile of the user (for example, testing a series of frequencies across the range of human hearing, such as 20 Hz to 20 kHz for example), or it could be a partial test profile of only a portion of the hearing range (such as 3-6 kHz, for example, which tends to be the range in which damage is most likely to occur from exposure to noisy environments).

The method includes delivering both a pre-pulse auditory stimulus and an auditory startle stimulus to the subject. The auditory startle stimulus is a loud sound sufficient to cause the subject to be startled and exhibit a startle response. Different subjects have differing startle-stimulus response functions, which are known or can be easily determined by one skilled in the art. Suitable startle stimulus typically include one or more audible wavelengths delivered at a volume of from 50 to 130 decibels (dB) sound pressure level (SPL). Lower sound levels are insufficient to stimulate a startle response, while higher sound levels can cause saturation, which decreases the effectiveness of the pre-pulse auditory stimulus. Accordingly, in some embodiments, a startle stimulus from 70 to 110 dB SPL, or from 80 to 100 dB SPL can be used. In some embodiments, the startle stimulus has a loudness of at least 70 dB SPL. Suitable audible wavelengths differ depending on the nature of the subject. For example, suitable audible wavelengths for a human subject range from 20 Hz to 20 kHz, while suitable audible wavelengths for a mouse subject range from 1 kHz to 70 KHz. Typically the startle stimulus will include multiple (e.g., wide-band) audible frequencies. The duration of the start stimulus can be from 5 to 100 milliseconds (ms), with some embodiments using a duration ranging 10 to 60 ms, and other embodiments using a duration ranging from 15 to 30 ms.

A pre-pulse auditory stimulus is delivered to the subject prior to delivery of the auditory startle stimulus. Pre-pulse auditory stimulus are administered at a single frequency (i.e., a pure tone), and a lower volume relative to the startle stimulus. The frequency of the pre-pulse auditory stimulus can be any wavelength audible to the subject, but should overlap with the wavelengths used in the startle stimulus. The pre-pulse auditory stimulus can be delivered at a volume from 10 to 80 dB SPL, while in some embodiments it is delivered at 10 to 70 dB SPL, while in further embodiments the pre-pulse auditory stimulus is delivered at 10 to 60 dB SPL. The duration of the pre-pulse stimulus can be from 5 to 100 milliseconds (ms), with some embodiments using a duration ranging from 10 to 60 ms, and other embodiments using a duration ranging from 15 to 30 ms.

In some embodiments, the method is repeated using pre-pulse auditory stimulus at a plurality of different audible frequencies. This allows for frequency-specific changes (e.g., loss) of hearing to be identified. Repeating the method involves carrying out all of the steps of the method, but using a pre-pulse auditory stimulus having a different audible frequency from what was used previously. For example, the method can be repeated 1, 2, 3, 4, 5, 6, 7, 8, 9, 10, or more than 10 times using different audible frequencies.

The method includes allowing a stimulus gap to pass after delivering a pre-pulse auditory stimulus to the subject, and before delivering an auditory startle stimulus to the subject. The stimulus gap is simply a period of time in which no auditory stimulus is delivered to the subject, in order to distinguish the pre-pulse auditory stimulus from the auditory startle stimulus. The gap should be long enough to distinguish the signals, but not so long that the pre-pulse inhibitory effect is lost. For example, the stimulus gap can have a value in the range of 10 ms to 200 ms. In some embodiments, the stimulus gap has a value in the range of 20 ms to 160 ms, while in further embodiments the stimulus gap has a value in the range of 40 ms to 120 ms. In some embodiments, the pre-pulse auditory stimulus can inhibit the startle response even if the gap is absent, so in some embodiments the stimulus gap can have a value in the range of 0 ms to 200 ms.

The method also includes measuring the startle response of the subject, and determining if the startle response of the subject to the auditory startle stimulus has decreased compared with a control value for the startle response. The startle response is a largely unconscious physical defensive response to sudden or threatening stimulus, such as sudden noise or movement. The onset of the startle response is a startle reflex reaction. The startle reflex is a brainstem reflectory reaction (reflex) that serves to protect vulnerable parts, such as the back of the neck (whole-body startle) and the eyes (eyeblink). There are many various reflexes that can occur simultaneously during a startle response. The fastest reflex recorded in human subjects happens within the masseter muscle or jaw muscle. Electromyography showed the latency response or the delay between the stimulus and the response recorded was found to be about 14 milliseconds. The blink of the eye which is the reflex of the orbicularis oculi muscle was found to have a latency of about 20 to 40 milliseconds. Out of larger body parts, the head is quickest in a movement latency in a range from 60 to 120 milliseconds. The neck then moves almost simultaneously with a latency of 75 to 121 milliseconds. Next, the shoulder jerks at 100 to 121 milliseconds along with the arms at 125 to 195 milliseconds. Lastly the legs respond with a latency of 145 to 395 milliseconds. This type of cascading response correlates to how the synapses travel from the brain and down the spinal cord to activate each motor neuron. Corresponding reflex reactions also occur in other types of subject, such as test animals.

The startle response can be measured in a variety of different ways. For example, when evaluating the start response in human subjects, a preferred measure of the startle response is detection of an eye blink. For evaluating test animals, the inventors used high-speed video recordings to visualize animal startles in order to identify a stereotyped waveform associated with a startle. Based on this information, an algorithm which automatically separates data into either startles or non-startle-related movement was developed. Accordingly, in some embodiments, a startle algorithm is used to measure the startle response of the subject. Mathematical analysis can also be used to normalize startle response magnitudes of individual animals to their body mass. For example, when evaluating the start response of test animals, a mathematical extrapolation is used to convert startle data (force) into center of mass displacement (height). Startle magnitudes measured in force (Newton) were converted to center of mass displacement (COMD; mm). Force was divided by body mass to yield instantaneous acceleration of the COM. The acceleration vector was then integrated to obtain instantaneous velocity and then integrated again to obtain the instantaneous position of the COM. The mathematical conversion normalizes for mass, allowing comparison between animals of different mass, and converts the forces sensed by a piezoelectric startle plate.

The method of detecting hearing loss further includes repeating the steps of delivering the pre-pulse auditory stimulus, allowing a gap of time to pass, delivering an auditory startle stimulus, and measuring the startle response, until the pre-pulse auditory stimulus delivered in that iteration of the method has a decibel value sufficient to cause a decrease in the startle response of the subject. A decrease can be identified by comparing the startle response with a control value for the startle response. A control value can be a startle response previously determined for the subject, or a typical startle response obtained by determining the average startle response from a number of subjects of the same species. When repeating the steps, the pre-pulse auditory stimulus should begin with a low decibel value, which increases each time the steps are carried out, until the pre-pulse auditory stimulus decreases the startle response of the subject as a result of pre-pulse inhibition. The decibel value can be increased in any manner when repeating the steps of the invention. For example, the decibel value can increase by 2, 5, or 10 decibels per iteration of the steps of the method. The decibel level of the pre-pulse auditory stimulus can start, for example, at 40 decibels, followed by pre-pulse auditory stimuli at 45 db, 50 db, 55 db, 60 db, and so forth, until a decrease in the startle response is detected.

Once a decrease in the startle response has been detected, one can determine whether or not the subject has suffered hearing loss. A subject can be determined to have hearing loss if the pre-pulse auditory stimulus required to decrease the startle response is higher than a pre-pulse auditory stimulus threshold value for the subject. Methods of determining a pre-pulse auditory stimulus threshold value for a subject are described herein. In embodiments where pre-pulse auditory stimuli having a variety of different frequencies are used, the method can also be used to characterize the specific frequency or frequencies of the hearing loss.

In some embodiments, the degree of hearing loss is determined. The degree of hearing loss correlates with the degree of difference between the pre-pulse auditory stimulus that caused a decrease in the startle response, and the pre-pulse auditory stimulus threshold value for the subject. Hearing loss can be classified by characterizing the hearing loss in different grades (e.g. “slight”, “moderate” or “severe”) in different frequency ranges (e.g. low-tone range, medium tone range, high-tone range). A specific hearing-loss class can thus be determined by a moderate loss of hearing in the low-tone range, a slight hearing loss in the medium frequency range and a severe hearing loss in the high-tone range. Other differentiating features of different hearing-loss classes are however also possible. For example the class of hearing loss can be characterized based on susceptibility to different forms of treatment; e.g., hearing loss that can be suitably treated only by a hearing aid worn behind the ear with a customized ear adapter piece, or the hearing-loss class a characteristic of which is that an open treatment to compensate for the hearing loss is also suitable.

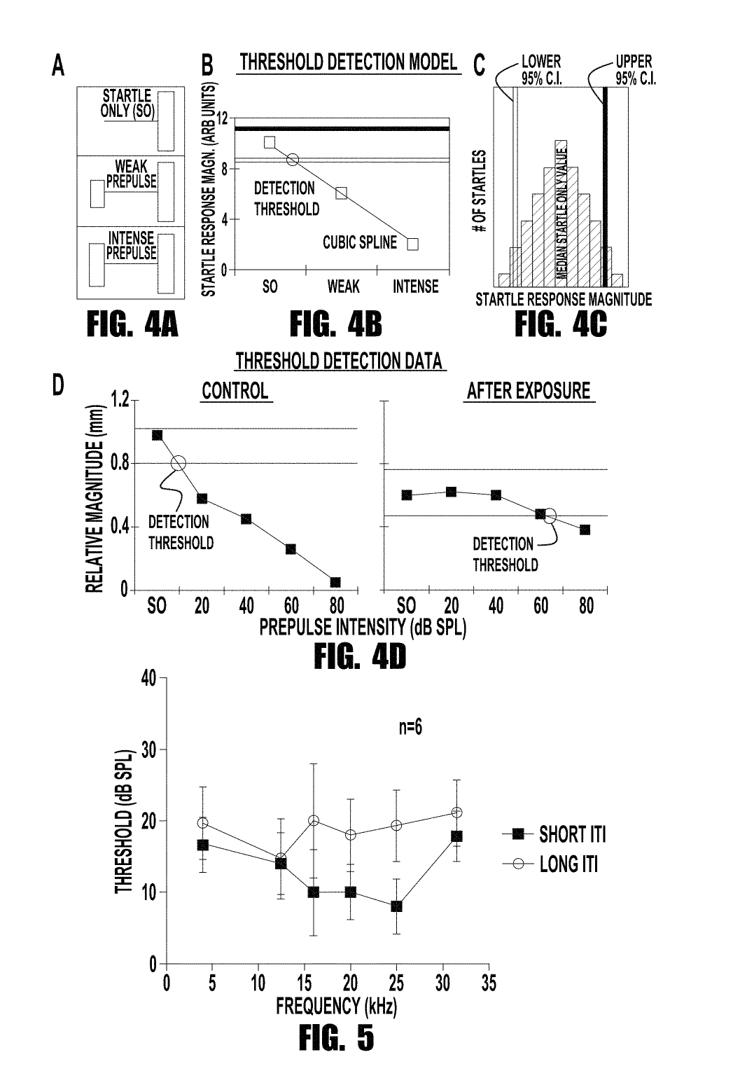

In some embodiments, the data collected carrying out a method of the invention is statistically transformed. A square root transform was found to be the best at generating a normal distribution of startle responses within each trial type, as assessed using the Anderson-Darling test. This standard statistical transform reduces skew by enhancing the lesser and reducing the larger startle magnitudes. See FIG. 4, which shows the results of statistically transforming the data obtained from PPI analysis. In some embodiments, a detection function can be calculated by fitting a cubic spline from the median transformed SO magnitude through the median transformed magnitudes for prepulses presented at various intensities.

In some embodiments, a visual display by means of which the result of the hearing test is shown to the subject and/or the test giver can also be used. The display can provide numeric or qualitative values. For example, the illumination of a red light can be used to indicate that the subject has severe hearing loss. If, on the other hand, an orange-colored light illuminates, this indicates that the subject has moderate hearing loss. A yellow-colored light can indicate that the subject has slight hearing loss, while a green light can, for example, indicate that there is no significant hearing loss present and no hearing aid is necessary. Naturally, there are many other possibilities in addition to the indications already mentioned to indicate the results of hearing tests by visual (e.g. by means of a display), acoustic (e.g. by means of voice output) or tactile means.

A subject, as defined herein, is an animal, preferably a mammal such as a domesticated farm animal (e.g., cow, horse, pig), a pet (e.g., dog, cat), or a non-human test animal (e.g., a rat, mouse, or primate). More preferably, the subject is a human (e.g., a human infant).

In some embodiments, the subject being evaluated is a subject that has been identified as having an increased risk of suffering from hearing loss. Hearing loss is mainly caused by damage to the cochlear hair cells, the sensory cells in the cochlea. Cochlear hair cell damage can result from a number of causes, including age-related damage or loss (presbycusis), acute or chronic noise exposure, acute or chronic drug exposure, e.g., aminoglycoside antibiotics and anti-cancer therapeutics, infections, syndromic and non-syndromic genetic mutations, and autoimmune disease.

In some embodiments, the method of detecting hearing loss can be used to identify and/or guide the treatment of subjects having hearing loss. Treatments for hearing loss include electronic cochlear implants, hearing assistive technology, audiologic rehabilitation, the use of hearing aids, and small molecule compounds for treating hearing loss. See U.S. Patent Publication No. 2015/126507.

In some embodiments, a plurality of subjects are evaluated. In this case, the method of the invention is carried out as described on more than one subject. This can be more readily done because of the relative speed of the method, and the fact that subjects can be evaluated without the need for a conscious response by the subject. For example, the method can be used to evaluate the hearing of 2, 3, 4, 5, 6, 7, 8, 9, 10, 15, 20, 25, 30, 35, 40, 45, 50, 60, 70, 80, 90, 100, or more than 100 subjects. In further embodiments, the method is used to evaluate a plurality of subjects at the same time; i.e., simultaneously.

The ability to evaluate a plurality of subjects simultaneously, without requiring a conscious response by the subject, also renders the invention well suited for screening subjects such as test animals that have been exposed to a drug. Accordingly, in some embodiments, the method further comprises administering a test drug to the subject before delivering a pre-pulse auditory stimulus to the subject. The test drug can be any drug which needs to be tested for having an effect (such as an undesired side-effect) on a subject's hearing.

Another aspect of the present invention is shown in FIG. 2, which provides a flowchart showing a method of determining a pre-pulse auditory stimulus threshold value for a subject 30. The pre-pulse auditory threshold value is a useful loudness value for a subject which can be used to evaluate the hearing of a subject using pre-pulse inhibition of the startle response. The method includes the steps of delivering a pre-pulse auditory stimulus to the subject 32, allowing a stimulus gap to pass 34, delivering an auditory startle stimulus to the subject 36, measuring the startle response of the subject 38, and identifying the pre-pulse auditory stimulus as a pre-pulse auditory stimulus threshold value for the subject when the measured startle response is lower than a control value for a startle response 40.

The pre-pulse auditory stimulus, the stimulus gap, the auditory startle stimulus, and the startle response have the characteristics described herein. Accordingly, steps 32, 34, 36, and 38 of FIG. 2 correspond to the same methods described for steps 12, 14, 16, and 18 of FIG. 1. However, while FIG. 1 shows a method of detecting hearing loss, FIG. 2 shows a method of determining a pre-pulse auditory threshold value. The primary difference is that the method of FIG. 2 is directed to identifying the pre-pulse auditory stimulus threshold value by determining when a change in the startle response occurs, whereas FIG. 1 is directed towards a method of determining if a change in the pre-pulse auditory stimulus value has occurred, which indicates that there has been a change in the subject's hearing. In some embodiments, the method is repeated using pre-pulse auditory stimulus at a plurality of different audible frequencies, which in other embodiments the startle stimulus has a loudness of at least 70 decibels sound pressure level.

In some embodiments, the method is used to determine a plurality of pre-pulse auditory stimulus threshold values for a plurality of subjects, which in further embodiments the plurality of pre-pulse auditory stimulus threshold values are determined simultaneously. The methods can be used to determine the pre-pulse auditory stimulus threshold in subjects that cannot give a conscious response, such as a non-human test animal or a human infant.

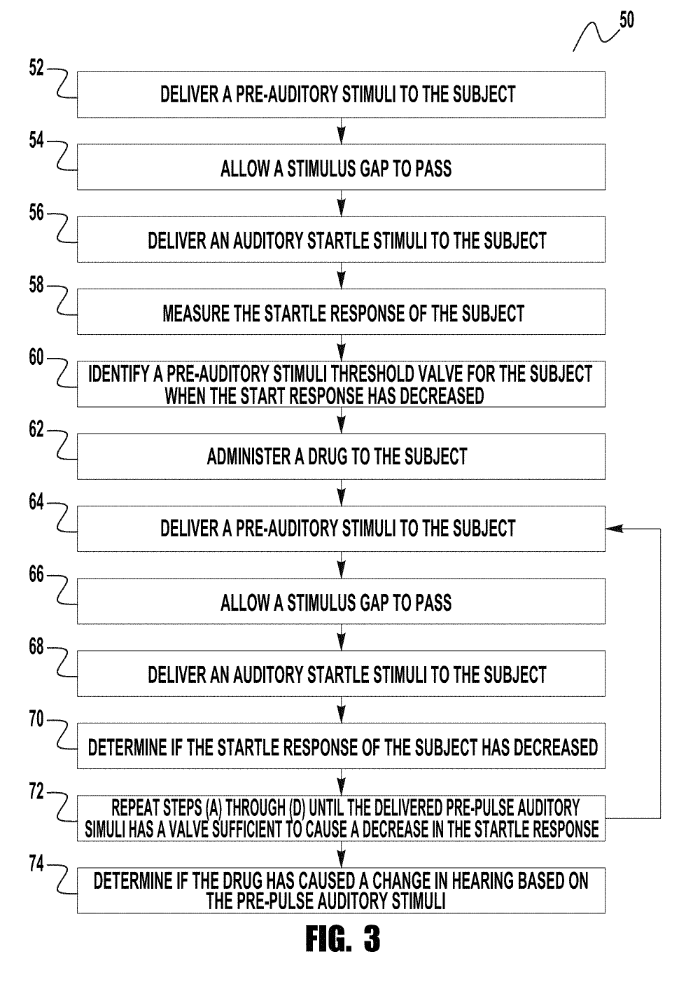

Another aspect of the present invention is shown in FIG. 3, which provides a flowchart showing a method of determining the effect of a drug on the hearing of a subject 50. The method includes both determining the pre-pulse auditory stimulus threshold value for the subject shown in FIG. 2, and then using the pre-pulse auditory stimulus threshold value to determine if the hearing of the subject has decreased or improved in response to administration of a drug, corresponding to the method shown in FIG. 1. While the method shown in FIG. 3 is directed to determining the effect of a drug on the hearing of a subject, this method can also be used to test the effect of other events on the hearing of a subject by replacing the step of administering the drug with the step of allowing the event of interest to occur.

The method of determining the effect of a drug on hearing includes first determining a pre-pulse auditory stimulus threshold value for the subject. This includes the steps of delivering a pre-pulse auditory stimulus to the subject 52, allowing a stimulus gap to pass 54, delivering an auditory startle stimulus to the subject 56, measuring the startle response of the subject 58, and identifying a pre-pulse auditory stimulus threshold value for the subject when the measured startle response is lower than a control value for the startle response 60.

Once the pre-pulse auditory stimulus threshold value has been determined for a normal subject, a drug is then administered to the subject 62. The drug can be administered to the subject using routine methods such as oral or intravenous administration, and can be administered together with a pharmaceutically acceptable carrier. In some embodiments, the drug is being screened to determine if it might be useful for treating hearing loss, while in other embodiments the drug is being screened to determine if it has an adverse effect on hearing. Examples of compounds that can cause hearing loss include some aminoglycoside antibiotics and anti-cancer therapeutics.

After the drug has been administered, the remaining steps of the method are carried out to determine the effect of the drug on hearing of the subject. These steps include delivering a pre-pulse auditory stimulus to the subject 64, allowing a stimulus gap to pass 66, delivering an auditory startle stimulus to the subject 68, measuring the startle response of the subject, and determining if the startle response of the subject to the auditory startle stimulus has decreased compared with a control value for the startle response 70, repeating steps 64 to 70 until the pre-pulse auditory stimulus administered has a value sufficient to cause a decrease in the startle response of the subject, and determining that the drug has caused hearing loss in the subject if the pre-pulse auditory stimulus required to decrease the startle response is higher than the pre-pulse auditory stimulus threshold value for the subject or determining that the drug has caused hearing improvement in the subject if the pre-pulse auditory stimulus required to decrease the startle response is lower than the pre-pulse auditory stimulus threshold value for the subject 74.

In some embodiments, the method is used to determine a plurality of pre-pulse auditory stimulus threshold values for a plurality of subjects. In other embodiments, the subject is a non-human test animal. In further embodiments, the method is repeated using pre-pulse auditory stimulus at a plurality of different audible frequencies.

An example has been included to more clearly describe a particular embodiment of the invention and its associated cost and operational advantages. However, there are a wide variety of other embodiments within the scope of the present invention, which should not be limited to the particular example provided herein.

### Example

### Prepulse Inhibition of the Acoustic Startle Reflex Vs. Auditory Brainstem Response for Hearing Assessment

Although PPI audiometry has several advantages over other currently used hearing assessment methodologies, several important questions need to be explored before it can be widely applied. First, it is still unknown whether it is sensitive enough to assess hearing in individual animals, which would be much more beneficial than group averages. Second, the extent of PPI threshold reliability from day to day is also unknown. Third, it is important to know whether PPI audiometry can be used to detect permanent threshold shifts caused by the most common hearing insult, noise-induced hearing loss. The goal of this study was to address these questions and compare PPI audiometry with the traditional ABR approach.

### Methods

Animals

A total of 16 male CBA/CaJ mice obtained from Jackson Laboratories were used. To avoid startle variability which is known to result from hormone fluctuations of the estrous cycle, female mice were not used in this study. Plappert et al., Physiology & Behavior 84: 585-594 (2005). This phenomenon has also been shown in human subjects (Kumari et al., Schizophrenia Research, 69: 219-235 (2004)). Mice were 12 weeks old at the beginning of the experimental procedures. They were housed in pairs within a colony room with a 12-h light-dark cycle at 25° C.

Ten mice were sound exposed as described below while six unexposed mice were used as controls. The exposed mice were tested during a 3 month period to detect permanent threshold shifts. The 6 control mice were used to test for the consistency of PPI measurements across time.

Acoustic Trauma

Mice were anesthetized with an intramuscular injection of a ketamine/xylazine mixture (100/10 mg/kg). An additional injection (50% of the initial dose) was given 30 min after the initial injection. Mice were unilaterally exposed to a one octave narrow-band noise centered at 12.5 kHz (˜8-17 kHz). This noise was generated using a waveform generator (Tektronix AFG 3021B), amplified (QSC RMX 2450) to 116 dB SPL, and played through a speaker (Fostex T925A Horn Tweeter). The output of the loudspeaker was calibrated with a 0.25-in. microphone (Brüel and Kjaer 4135) attached to a measuring amplifier (Brilel and Kjaer 2525) and found to be ±4 dB between 4 and 60 kHz. During exposure the speaker was located ˜5 cm from the animal's right ear. The left external ear canal was obstructed with a cotton plug and a Kwik-Sil silicone elastomer plug (World Precision Instruments), a manipulation which typically reduces sound levels by 30 to 50 dB SPL. Turner et al., Behavioral Neuroscience 120, 188-195 (2006).

Auditory Brainstem Response Testing

Mice were anesthetized with ketamine/xylazine as during the acoustic trauma. Sterile, stainless-steel recording electrodes (connected to a Tucker Davis Technologies (TDT) RA4LI Low Impedance Headstage) were placed subdermally, one behind each pinna with the reference electrode along the vertex. Tone bursts at 4, 12.5, 16, 20, 25, and 31.5 kHz were presented at increasing sound intensities ranging from 10 to 80 dB SPL in 10 dB steps. Tones were 5 ms duration, 0.5 ms rise/fall time and delivered at the rate of 50/s. ABRs were averaged over 300 repetitions. These waveforms were amplified (TDT RA4PA Medusa Preamplifier), digitized (TDT RZ6 Multi-I/O Processor), and analyzed offline using custom made software (TDT OpenEx). Thresholds, the smallest sound amplitude that evoked a visible ABR, were determined by visually examining the ABR waveforms in response to every sound frequency presented at different sound levels. ABR wave one amplitudes (μV peak to peak) were measured at each intensity/frequency combination for all exposed mice at all time points tested (prior to exposure (control), one day after exposure, and three months after exposure).

Acoustic Startle Hardware/Software

The equipment used to collect all acoustic startle data has been described in detail previously. Longenecker R J, Galazyuk A V, Brain Research 1485, 54-62 (2012). Commercial hardware/software equipment from Kinder Scientific, Inc. was used in behavioral experiments. Each behavioral testing station was lined with anechoic foam to prevent sound reflection and wave cancelling sound echoes (Sonex foam from Pinta Acoustics). A small customization of the hardware's startle stimulus system was made by adding SLA-4 (ART) amplifiers to adjust sound levels to correct for variations in speaker loudness between testing station. Mice restrainers were open walled to allow for maximum sound penetration. Background sound levels within each testing chamber were calibrated with a 0.25-in. microphone (Brüel and Kjaer 4135) attached to a measuring amplifier (Brüel and Kjaer 2525) and found to be less than 40 dB SPL between 4 and 60 kHz. Startle waveforms were recorded using load cell platforms which measure actual force changes during an animal's jump. Each load cell was calibrated with a 100 g weight which corresponds to 1 newton of force. Offline waveform analysis converted these forces into center of mass displacement (in mm). Grimsley et al., J Neurosci Methods 253:206-217 (2015).

Reflex Modification Audiometry: Prepulse Detection Testing

Testing sessions contained two types of stimulus. First, a startle stimulus (wide-band noise, 100 dB SPL, 20 ms in duration, 1 ms rise/fall) was presented alone; this is referred to in the text as startle only (SO) (FIG. 4A). The second stimulus type consisted of a congruent startle stimulus preceded by a prepulse (FIG. 4A). Prepulse stimulus were 20 ms pure tones with a 1 ms rise/fall time presented at six different frequencies (4, 12.5, 16, 20, 25, and 31.5 kHz) 100 ms before the startle stimulus. These prepulses were played in a pseudo-randomized range of intensities (10-80 dB SPL, 10 dB step) for a given sound frequency. Each frequency/intensity combination was presented 39 times. SOs were pseudo-randomly mixed throughout each testing session. In preliminary experiments, inter-trial intervals (ITIs) were randomized between 15 and 25 seconds. With this ITI, a complete testing period (1872 prepulse trials, and 468 startle alone trials) required 15 hours with a 1 hour break for the animal to rest between hour 7 and 8. In order to shorten the testing period to reduce stress on the animals and to allow similar time constraints as ABR testing, we tested animals at short ITIs of 4-6 s. In this condition, a complete testing session, in which 8 mice could be tested simultaneously, lasted roughly 1.5 hours. Interestingly, when the same mice were tested in both ITI conditions, response thresholds were lower when the shorter ITIs were used (FIG. 5). Following this experiment, all remaining PPI testing was completed at 4-6s ITIs. Startle-only responses were closely monitored throughout testing sessions to ensure that startle magnitudes were not significantly affected by habituation as described in detail below. Additionally, it has been shown that unlike rats, many strains of mice show little to no reduction in their acoustic startle reflex magnitude throughout testing sessions. Bullock et al., Behavioral Neuroscience 111 (6): 1353-1360 (1997). This is an important factor when assessing prepulse inhibition of the acoustic startle reflex across sessions, and experimental conditions.

Reflex Modification Audiometry: Startle Waveform Identification and Measure

All waveforms collected during testing sessions were analyzed offline using a recently developed automatic method of startle waveform identification via a template matching paradigm. Grimsley et al., J Neurosci Methods 253:206-217 (2015). In this recent study we used high-speed video recordings (1,000 frames/s) to visualize animal startles in order to identify stereotyped waveforms associated with a startle. This allowed us to develop custom software which automatically separates data into either startles or non-startle-related movements. Based on this separation, we have only included trials that resulted in successful startle responses in our data analysis. We also used a mathematical approach to normalize startle response magnitudes of individual animals to their body mass. This mathematical conversion normalizes for mass, allowing legitimate comparisons between animals of different mass.

Reflex Modification Audiometry: Prepulse Detection Threshold Identification

In each session, the magnitudes of the SOs were compared to the magnitudes of startles preceded by prepulses with various frequency and intensity (FIG. 4A). A significant reduction in the magnitude of the startle response by the prepulse compared to the SO was defined as the prepulse detection threshold (FIG. 4B). Identifying this threshold involved several steps. First, we examined the distribution of the raw startle magnitudes. Startle magnitude is known to be quite variable in mice, and indeed the startle magnitude typically was strongly positively skewed. This positive skew could allow aberrantly high values to obscure reliable changes in startle magnitude across stimulus parameters. Furthermore, a normal distribution of magnitudes is an assumption underlying the method for threshold determination that we use here. Thus we transformed the data. A square root transform was found to be the best at generating a normal distribution of startle responses within each trial type, as assessed using the Anderson-Darling test. This standard statistical transform reduces skew by enhancing the lesser and reducing the larger startle magnitudes. Second, for each animal, the transformed SO data was bootstrapped to determine 95% confidence intervals for the SO response magnitudes (FIG. 4C). Then we calculated medians of transformed magnitudes for each frequency and intensity, as the median value is a better measure of central tendency than the mean for skewed distributions. For each frequency, a detection function was calculated by fitting a cubic spline from the median transformed SO magnitude through the median transformed magnitudes for prepulses presented at various intensities. Detection threshold was defined as the sound level at which the fitted detection function crossed the lower 95% confidence interval (FIG. 4B). The detection threshold increases after sound exposure because the slope of the detection curve becomes “flatter,” likely due to hearing loss (FIG. 4D).

Statistical Analyses

Both ABR threshold data and PPI thresholds were evaluated with a repeated measures design because the same animals were used in each condition. Different frequencies (six) and conditions (three) were used as independent variables in the repeated measures analysis of variance (ANOVA). Planned comparisons between specific frequency/intensity combinations were assessed with Fisher's Least Significant Difference (LSD) post-hoc tests. Linear regressions were used to evaluate correlations between PPI and ABR data. Values throughout the manuscript are specified as means or medians±standard error of the mean (SEM). We used an alpha level of 0.05 for all statistical tests. P values significant at the 0.05 level are indicated by one symbol while those significant at the 0.001 level or lower are indicated by two symbols. Due to the complexity of some figures, various symbols have been used to represent significant differences: *=significant difference between the control condition and one day after exposure; #=significant difference between the control condition and three months after exposure; @=significant difference between one day after exposure and three months after exposure.

Results

Consistency of PPI Audiometry

Accuracy and precision are crucial for any methodology that is assessing hearing loss. As such, we tested whether thresholds identified with PPI audiometry were comparable to thresholds determined by behavioral audiograms. Radziwon et al., J. Comp. Physiol. A. Neuroethol. Sens. Neural Behay. Physiol. 195(10), 961-969 (2009). We also assessed both the variation between mice as well as the variation within individual mice across multiple testing sessions. On three consecutive days of testing, thresholds at all frequencies tested (4, 12.5, 20, 25, 31.5 kHz) were below 40 dB SPL with inter-animal differences of usually less than 10 dB (FIG. 6). Importantly, these data confirmed results from a previous prepulse modulation study conducted on humans. Reiter L A, J Speech Hear Res., 24(1), 92-98 (1981). The variations in PPI thresholds for individual mice across days of testing and different frequencies did not exceed 5.77 dB (FIG. 6A-F). Interestingly, the Reiter study found that audiograms collected by the startle reflex are more “flattened” across frequency in comparison to more traditional audiograms, which is confirmed by our data in mice (FIG. 6, 7A-C control).

Comparing PPI and ABR Audiometric Thresholds for Hearing Assessment

To determine if PPI audiometry is sensitive to both temporary and permanent threshold shifts we exposed 10 mice to a one octave narrowband noise centered at 12.5 kHz and presented at 116 dB SPL for 1 hour. Immediately after exposure, PPI thresholds were dramatically increased which resulted from a “flatter” cubic spline function in comparison to the “steep” monotonic function in the control condition, thus contributing to increased detection thresholds (FIG. 4D).

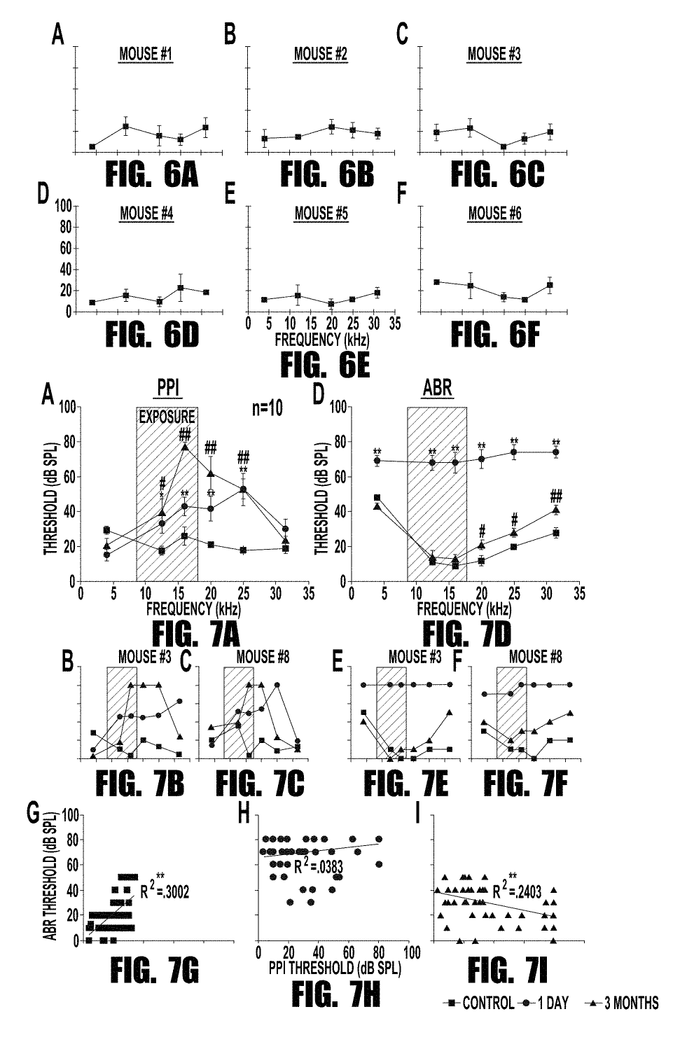

The effect of noise-induced trauma on hearing was evaluated with PPI and ABR thresholds collected before, one day, and three months after exposure. A repeated measures ANOVA found that both PPI and ABR thresholds were significantly elevated one day (PPI: F(1,53)=75.8, p<0.001; ABR: F(1,53)=18.3, p<0.001) and three months following exposure (FIG. 7A, D). Least Significant Difference posthoc pairwise comparisons checking for frequency specific differences found significant PPI threshold elevations at 12.5, 16, 20, and 25 kHz one day after exposure (FIG. 7A) (12.5 kHz: p<0.05; 16, 20, 25 kHz: p<0.001). ABR thresholds at almost all frequencies were maximally elevated one day after exposure (FIG. 7D) (4-31.5 kHz: p<0.001). Interestingly, at the three month time point PPI threshold differences remained elevated at 12.5 and 25 kHz (12.5 kHz: p<0.05; 25 kHz: p<0.001) yet were further elevated at 16 and 20 kHz (16-20 kHz: p<0.001), the frequencies directly surrounding the spectral characteristics of the acoustic trauma (FIG. 7A). However, ABR thresholds nearly recovered to baseline levels for most frequencies at three months, while some of them remained statistically elevated (FIG. 7D) (20-25 kHz: p<0.05; 31.5 kHz: p<0.001). Representative examples of PPI (FIGS. 7B, C) and ABR (FIGS. 7E, F) audiometric functions from two individual mice show typical patterns of damage. One day after exposure both mice demonstrated broad temporary threshold shifts. Mouse #3 and #8 showed PPI deficits in the range of 12.5 to 31.5 kHz and 12.5 to 25 kHz, respectively (FIGS. 7B, C). These deficits roughly correspond to the non-specific frequency deficits which were observed with ABRs (FIGS. 7E, F). At three months after exposure the results of hearing threshold assessments for both measurements were dramatically different. While PPI assessment showed narrow frequency specific deficits at the high edge of the range of exposure, ABR thresholds returned to near pre-exposed levels (FIG. 7D). Interestingly, despite nearly identical sound exposure, the individual mice exhibited deficits at slightly different frequency ranges (compare FIGS. 7B and C).

The relationship between PPI and ABR thresholds was directly compared with a linear regression for each condition. In the control condition (pre-exposure) PPI and ABR thresholds had a significant weakly positive correlation (R²=0.3 (F(1,57)=24.4, p<0.001)) (FIG. 7G). This suggests that these measures can be compared with some confidence in normal unexposed animals. However, one day after exposure this correlation disappeared (R²=0.038) (FIG. 7H), as might be expected by such drastic changes in ABR thresholds and only moderate changes in PPI thresholds. Interestingly, a weak negative correlation (R²=0.24 (F(1,57)=18, p<0.001)) was seen at three months after exposure due to the fact that significantly elevated PPI deficits were centered around the exposure frequencies whereas ABR thresholds recovered nearly to baseline levels (FIG. 7I).

Comparing PPI and ABR Amplitudes for Hearing Assessment

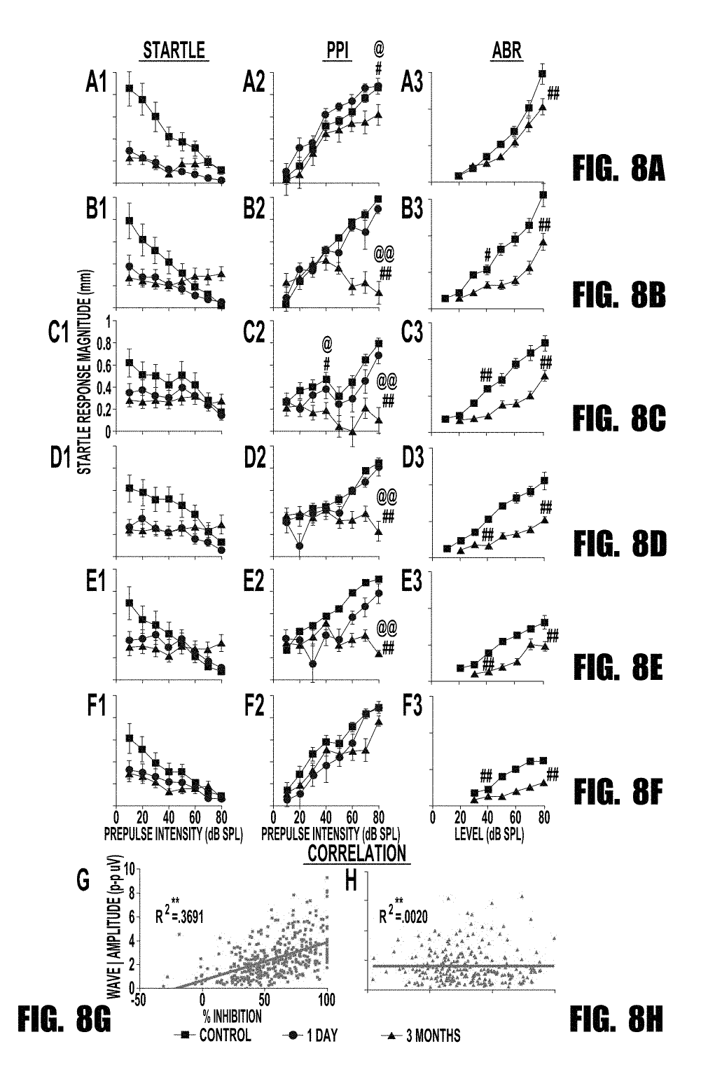

Recent studies have promoted the importance of examining ABR amplitudes instead of thresholds when evaluating noise induced hearing loss because of the strong link to the loss of suprathreshold auditory nerve fibers which are the most vulnerable during acoustic trauma. Kujawa S G, Liberman M C, J Neurosci 29, 14077-14085 (2009). For this reason we compared prepulse modulated startle magnitudes and the resulting inhibition (PPI) with wave one ABR amplitudes (FIG. 8) in the same mice that were tested for thresholds (FIG. 7). After exposure, a repeated measures ANOVA showed that prepulse modulated startle magnitudes were reduced significantly across all frequencies tested (4 kHz: F(1,64)=79.7, p<0.001; 12.5 kHz: F(1,64)=29.9, p<0.001; 16 kHz: F(1,64)=18.6, p<0.001; 20 kHz: F(1,64)=41.2, p<0.001; 25 kHz: F(1,64)=6.7, p<0.05; 31.5 kHz: F(1,64)=25.7, p<0.001) (FIG. 8A1-F1, 1 day post-exposure compared with pre-exposure). The differences were most pronounced for low intensity prepulses, when the startle stimulus was inhibited the least. This pattern for prepulse modulated startle magnitudes changed somewhat at three months, in that for most frequencies tested, the audiometric function flattened due to a decrease in startle magnitude at low intensity prepulse conditions, but an increase of startle magnitudes at high prepulse intensities (FIG. 4D; FIG. 8A1-F1). This might be explained by decreased startle values as shown later (FIG. 10). The prepulse inhibition for each prepulse modulated startle magnitude was calculated by dividing the SO trials by each prepulse trial. The resulting % inhibition curves show clear frequency-specific deficits most prominently expressed between 12.5 and 25 kHz only three months after exposure (FIG. 8A2-F2) (4 kHz: F(1,64)=34.7, p<0.001; 12.5 kHz: F(1,64)=52.93, p<0.001; 16 kHz: F(1,64)=58.68, p<0.001; 20 kHz: F(1,64)=612.84, p<0.001; 25 kHz: F(1,64)=433.773, p<0.001. Interestingly, these inhibition changes were mostly observed with high intensity prepulses, while low intensity prepulses remained mostly intact. The notable exception to this pattern was seen with inhibition at 16 kHz, in which low intensity prepulses also resulted in reduced inhibition from control levels (FIG. 8C2). In conjunction with prepulse modulated startle magnitudes and PPI changes, after exposure, a repeated measures ANOVA showed that ABR amplitudes were reduced significantly across all frequencies (FIG. 8A3-F3, 3 month post-exposure compared with pre-exposure) (4 kHz: F(1,72)=23.8, p<0.001; 12.5 kHz: F(1,72)=88.9, p<0.001; 16 kHz: F(1,72)=104.8, p<0.001; 20 kHz: F(1,72)=169.7, p<0.001; 25 kHz: F(1,72)=82.8, p<0.05; 31.5 kHz: F(1,72)=233.6, p<0.001). It is important to mention that wave one ABR amplitudes were not measured one day after exposure because no ABR response was present at that time, likely due to a very strong temporary threshold shift.

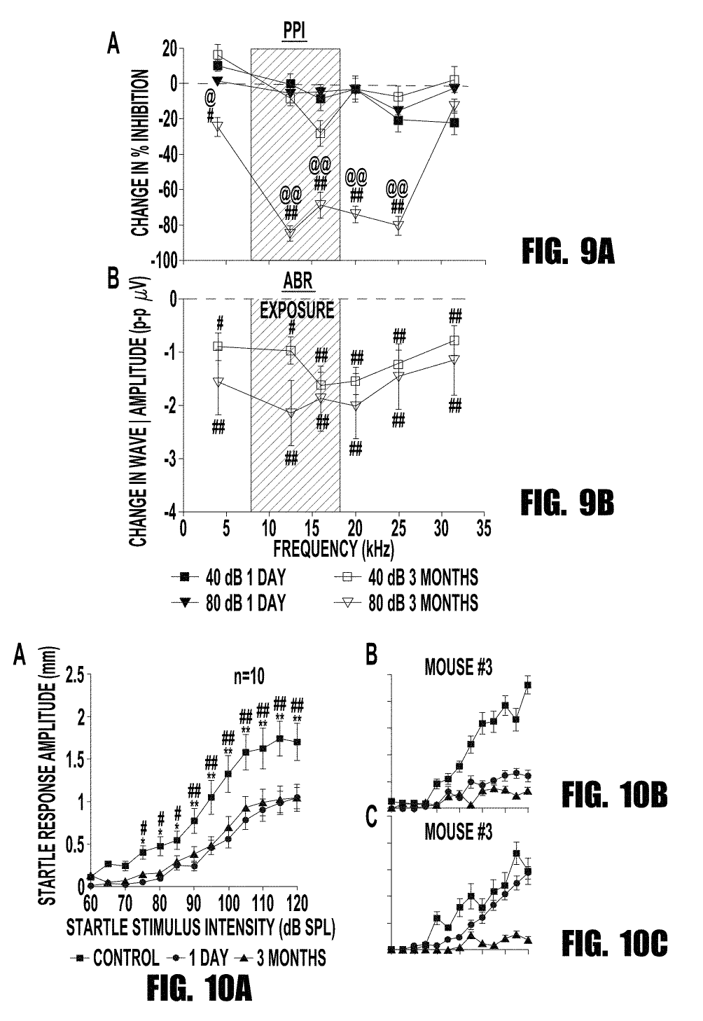

Interestingly, when PPI and ABR wave one amplitude deficits were plotted across frequencies as a function of difference from control amplitudes, unique patterns of damage were observed (PPI FIG. 8A2-F2, ABR wave one amplitudes FIG. 8A3-F3, compared in FIG. 9A, B respectively). Planned comparisons between testing epochs at 40 and 80 dB prepulse levels were assessed with LSD post hoc tests, for both ABR amplitudes and PPI. These intensities were chosen to test the contribution between high and low threshold auditory nerve fibers. ABR wave one amplitude deficits were centered at or around the upper end of the exposure frequency range, with 80 dB SPL wave one amplitudes significantly more affected by exposure than 40 dB SPL amplitudes (F(1,53)=75.3, p<0.001)(FIG. 9B). This is consistent with decreased synapse density for high threshold nerve fibers. PPI deficits are shown in FIG. 9A. One day after exposure (FIG. 9A), only minor differences in prepulse inhibition were observed in response to low (40 dB SPL) or high intensity prepulse stimulus (80 dB SPL). However, three months after exposure (FIG. 9A), the 40 dB SPL prepulse modulation was nearly identical to the one day after exposure, whereas the high intensity 80 dB SPL prepulse showed large magnitude increases (FIG. 9A square vs triangles), again suggesting severe hearing loss as a result of decreased synapse density from high threshold nerve fibers.

The relationship between PPI and ABR wave one amplitudes was directly compared with a linear regression for each condition. A very similar pattern was seen between PPI/ABR amplitude correlations (FIG. 8G, 5H) and threshold correlations (FIG. 7G, I). In the control condition (unexposed) PPI and ABR amplitudes had a significant weakly positive correlation (R²=0.3691 (F(1,478)=279.81, p<0.001)) (FIG. 8G). Generally, increasing inhibition (as a result of increasing prepulse intensities) followed a pattern similar to ABR wave one amplitudes at corresponding stimulus intensities. However, three months after exposure this correlation (R²=0.0200), was weakened substantially (F(1,478)=0.930, p=0.335) (FIG. 8H). This can be explained by the fact that inhibition to low intensity prepulses at the three month time point generally followed control levels of inhibition, while inhibition to high intensity prepulses was reduced markedly, as described above. Overall, the amplitude assessments between PPI and ABR tests differentially correlate based on condition, but are generally predictive of one another in control conditions.

Effects of Sound Exposure on Startle Magnitude and Probability

The magnitude of the startle response is often decreased either after sound exposure (Longenecker et al., SpringerPlus 3, 542 (2014); Lobarinas et al., Hearing Research 295, 150-160 (2013)) or due to habituation. Both may confound experimental findings by causing a “floor effect”. To test for this possibility, startle-only input/output functions were collected before, one day after, and three months after sound exposure for the group of ten exposed mice (FIG. 10A). Since our PPI testing used a 100 dB SPL startle stimulus intensity, we wanted to determine if this startle intensity could evoke a sufficiently strong startle response to ensure that it could still be inhibited by a prepulse. Although the startle response amplitude was significantly lowered one day (F(1,104)=172.9, p<0.001), and three months (F(1,117)=97.7, p<0.001) after exposure, the startle magnitude remained more than 2 standard deviations above baseline of animal background movement, a concept we previously introduced. Longenecker R J, Galazyuk A V, Brain Research 1485, 54-62 (2012); FIG. 10A. Importantly, the damage due to sound exposure reduced the startle reflex to varying degrees in each mouse, which is important when considering both ABR related pathologies and prepulse inhibition (FIG. 10B, C). This can effectively reduce the dynamic range of inhibition and should be monitored closely in each animal. An example of this dynamic range change is shown in FIG. 4D.

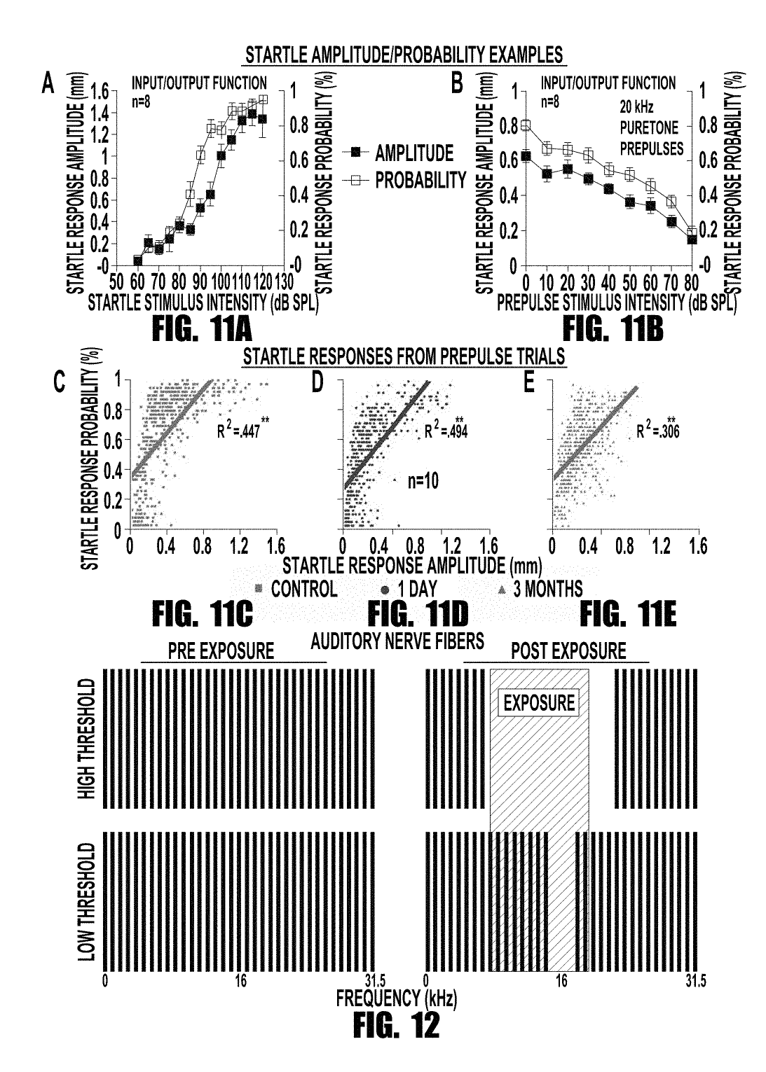

To further ensure that startle responses are properly assessed, our automatic startle waveform identification software provided us with an assurance that only true startle responses were included in our PPI data analysis (Grimsley et al., J Neurosci Methods 253:206-217 (2015)). This technology is especially important for separating small startle waveforms from non-stimulus-related movements. The percentage of non-startles is stable across the session. The startle probability is directly correlated with the startle magnitude, so if the startle magnitude is stable (lack of habituation), then the startle probability will also remain constant. As mentioned above, each stimulus (prepulse level or startle only) was presented 39 times per session. We found that the startle response magnitude changes that occur within input/output functions (FIG. 11A), or within prepulse modulation functions (FIG. 11B) are closely correlated to startle probability both in the control (R²=0.447 (F(1,478)=387, p<0.001)) and in exposed animals at one day (R²=0.494 (F(1,430)=421, p<0.001)) or three months (R²=0.306 (F(1,478)=213, p<0.001)) post exposure (FIG. 11C-E). This result is very robust across sessions, animals, and conditions. In FIG. 11A the startle probability elicited from a 110 dB SPL was about 80%. That implies that 20% of the trials were excluded because they were not determined to be startles by our waveform classifier system. This tool allowed us to not only accurately identify startle waveforms, but also to determine whether the startle response probability was constant or changing with prepulse parameters. This is most critical when the startle reflex is severely depressed as a byproduct of sound exposure, or when high intensity prepulses are presented.

Habituation to continually presented startle stimulus is also a concern during extended testing paradigms. To ensure habituation was not a significant confounding factor for the ASR in PPI, we monitored the startle magnitudes throughout the testing sessions. A Spearman correlation between startle magnitude and time of testing was run to identify whether magnitudes decreased during testing. The correlation was very small but significant (rs=0.146, p<0.001), suggesting that the startle response magnitudes were actually slightly elevated during recording sessions. This data is supported by previous findings that many strains of mice do not habituate to the acoustic startle reflex. Bullock et al., Behavioral Neuroscience 111 (6): 1353-1360 (1997). This has also been shown specifically for CBA/CaJ mice.

### DISCUSSION

We found that after several refinements, PPI audiometry was capable of measuring pre-pulse detection thresholds in mice much faster (90 minutes vs 9240 minutes) than previously shown in rats. Fechter et al., J. Acoustic. Soc. Am. 84(1), 179-185 (1988). It requires roughly as much time as standard ABR measurements, but can be conducted on multiple animals simultaneously without any anesthesia. Spectrally broad temporary threshold shifts, resulting from sound exposure, can be detected by PPI audiometry and are similar to those detected by ABRs (FIG. 7A, D) and behavioral audiograms. Heffner et al., J. Acoustic. Soc. Am. 124(2), 1093-1104 (2008). However, while high frequency permanent ABR threshold shifts were observed, lower frequency ABR thresholds recovered to baseline levels. This differed substantially from PPI thresholds which were elevated more broadly across frequencies and narrowed to substantial frequency-specific threshold shifts focused at the upper limit of the sound exposure frequencies. Importantly, PPI audiometry and ABR wave one amplitudes were both able to detect noise-induced hearing damage many months after sound exposure. Furthermore, PPI audiometry may reveal elements of higher-level plasticity in the auditory pathway, as discussed below. This suggests that each of these methods could be useful in assessing hearing related dysfunctions due to noise exposure, but might work better in conjunction, as they each are measuring different aspects of the auditory system.

What do ABRs and PPI Audiometry Measure?

The auditory brainstem response is by far the most popular methodology to assess the auditory system, as it can rapidly evaluate the functional connectivity of the auditory pathway. These recordings are able to identify gross malfunctions of the auditory system and are a useful tool for assessing the efficacy of sound trauma. The ABR is a non-invasive evoked potential usually conducted in anesthetized animals. It consists of five positive waves, each representing a synchronized neural output generated by auditory structures at and below the level of the inferior colliculus. Melcher J R, Kiang N Y S, Hearing Research 93, 52-71 (1996). This method allows for an in depth frequency specific synopsis of the cochlea and how it changes as a result of acoustic insults. A major advantage of this technique is that it can be conducted on humans, which allows comparison to animal models. Additionally, it can be done non-invasively and rather quickly, which allows screening of multiple animals in a short period of time. However, perhaps the biggest disadvantage of ABRs is the need for anesthesia in animal models which can alter the neural output in confounding ways. Additionally, ABRs can become more difficult to collect in older/larger animals due to increased fat to muscle ratios. Lastly, while it is known that ABRs can assess neural plastic changes that occur in the auditory brainstem, it is less certain if they directly measure neural computations above the level of the inferior colliculus. Sensorimotor gating experiments utilizing PPI of the acoustic startle reflex can also monitor neural plastic changes in these lower auditory structures but importantly, also have the ability to monitor cortical structures which are critically involved in modulating prepulse inhibition. Du et al., J Neurosci 31(38), 13644-13653 (2011). Thus, ABR audiometric functions provide some advantages, but may offer a more complete picture of the auditory system function if combined with a behavioral measure like PPI audiometry.

PPI audiometry is effective because the acoustic startle reflex (ASR) is greatly influenced by preceding stimulus. Hoffman H S, Searle J L. J Comp Physiol Psychol., 60(1), 53-58 (1965); Willott J F, Carlson S, Behavioral Neuroscience 109(3), 396-403 (1995). We have confirmed what was previously found, that prepulses presented at threshold levels can be used to assess an animal's hearing by modulating the startle motor output (FIG. 7A-C; 8A2-F2). Fechter et al., J. Acoustic. Soc. Am. 84(1), 179-185 (1988). However, before PPI audiometry can be used routinely for hearing assessment in laboratory animals, it is important to have a clear understanding of what underlying circuitry is being assessed.

The neuronal circuitry underlying the ASR is straightforward and has been studied extensively. Koch M, Prog Neurobiol 59(2), 107-28 (1999). The primary startle circuit includes the serial connections between the auditory nerve fibers, cochlear root neurons (in some species), and the nucleus reticularis pontis caudalis (PnC) region. The motor output is then sent to the interneurons of the spinal cord resulting in a startle. The simple relationship between evoked stimulus amplitude and the associated startle response magnitude (FIG. 10) and probability (FIG. 11) can be explained by the output of the nucleus reticularis pontis caudalis. Furthermore, it has been shown that if one ear is compromised (Gomez-Nieto et al., Front. in Neurosci 8(216): 1-18 (2014)), that the startle magnitude is reduced, as a direct result of the neural response inputs to the PnC. Interestingly, our study found that startle response magnitude and probability are considerably well correlated, which suggests that even in low startle magnitude conditions, which can be observed in animals with hearing loss (FIG. 11E), it is possible to include only valid startle data. This is important to consider when using PPI audiometry on sound-exposed animals. We controlled for the low startle confound in this study by using our advanced startle waveform analysis (Grimsley et al., J Neurosci Methods 253:206-217 (2015)) and by monitoring the probability of startles in various conditions (FIG. 11C-E).

When prepulses are added to the ASR construct, the neural circuitry is far more complex (Swerdlow et al., Psychopharmacology 156, 194-215 (2001)). The amount of inhibition that a prepulse has on the startle results from complicated neural computations that arise from various brain regions. The primary circuitry for mediating a prepulse's effect on a startle resides below the level of the colliculi. These nuclei send GABAergic projections that terminate at the nucleus reticularis pontis caudalis. Although this simple circuitry is directly responsible for generating the inhibitory effects on the startle, many higher nuclei can modulate these effects as well. Prepulse modulatory mechanisms are far more complicated and depend on behaviorally salient factors such as inter-stimulus interval, frequency, stimulus duration, prior auditory experience, and habituation. It has been shown that a large degree of top-down modulation can influence prepulses. Larrauri J, Schmajuk N, EXS, 98, 245-278 (2006). The amygdala, hippocampus, prefrontal cortex, auditory cortex and many other structures have been flagged as the source of this top-down modulation. To this extent, it seems that PPI audiometry has the potential to assess large portions of the auditory neuraxis and other neocortical regions. Therefore it is likely that PPI audiometry could be a useful tool to assess neuroplastic changes in awake behaviorally responsive animals. PPI audiometry would be especially suited for labs that already employ the ASR for other purposes such as tinnitus detection (Turner et al., Behavioral Neuroscience 120, 188-195 (2006)) or psychopharmaceutical studies. Phillips et al., J Psycopharmacol; 14(1):40-5 (2000). It should be noted that PPI tests are performed on multiple animals concurrently with no prerequisite training for the animals or the handlers. In this study we found that we could assess auditory thresholds at 6 different frequencies in less than two hours, and future work will aim to reduce this length of time even further. This rate of testing would be exceptionally valuable for drug testing which requires large sample sizes.

Comparison of PPI Audiometry to ABR Assessments

Comparing PPI and ABR Thresholds

The ASR has been shown to be greatly influenced by preceding stimulus and can be used for hearing assessment in laboratory animals and humans. In animals PPI can provide a behaviorally assessed, high throughput solution to assess auditory thresholds in normal hearing and sound exposed animals (FIG. 10A-C). To this point, however, the reliability of PPI audiometry to obtain thresholds across animals and days has not been tested. Previous work has shown that prepulse inhibition has only small variations from day to day (Willott et al., Behavioral Neuroscience 117(4), 716-727 (2003)), corroborating with our data showing consistency in PPI audiometry across animals and days (FIG. 9). Secondly, it is important to note that there were some differences in the audiogram generated by our PPI method and previous behavioral assessments in animals. Radziwon et al., J. Comp. Physiol. A. Neuroethol. Sens. Neural Behay. Physiol. 195(10), 961-969 (2009). Although most of the thresholds matched within 10 dB SPL of behavioral paradigms, we notice that for some animals the 4 kHz threshold was much lower. This finding however was also seen in humans Reiter L A, J Speech Hear Res., 24(1), 92-98 (1981). It is possible that the sounds inhibiting the startle reflex do not reflect the same circuitry as is involved with behavioral decision-making processes that lead to behavioral thresholds. Finally, we evaluated whether PPI could detect temporary and permanent threshold shifts due to noise exposure. Walter found that sound exposure caused increases in both PPI and ABR related thresholds, which is in good agreement with our data (FIG. 10A-F). Walter et al., Open Journal of Acoustics 2, 34-49 (2012). However, it is well known that permanent threshold shifts will develop over some time after exposure and may have very different spectral characteristics than temporary threshold shifts. Three months after exposure ABR thresholds recovered to near baseline levels (FIG. 10D-F) (Kujawa S G, Liberman M C, J Neurosci 29, 14077-14085 (2009)), whereas we found that PPI threshold became further increased at frequency specific regions of sound exposure compared with thresholds one day after exposure (FIG. 7A-C). This suggests that PPI thresholds are detecting some hearing deficits that ABR thresholds are not. Since top down PPI modulation of the startle reflex can come from auditory structures above the inferior colliculus (the furthest reach of ABRs), it is possible that changes in PPI audiometric thresholds represent higher order deficits as a result of sound exposure. It is well known that tonotopic maps of neuronal activity undergo plastic changes in response to sound exposure. Wang et al., Neuroscience 164, 747-759 (2009). Such reorganization may account for the poor correlation between PPI and ABR thresholds one day after exposure (FIG. 7H), and the negative correlation three months after exposure (FIG. 7I). However, it is possible that the precision of these correlations was not ideal, because while ABR thresholds are measured on a 10 dB interval scale, PPI thresholds were determined using a ratio scale via the cubic spline function (all rational numbers between 0-80). Despite this fact, that we are comparing two very distinct measures of hearing, correlations between these two hearing assessments are generally predictive of one another in control conditions (FIG. 7G).

Comparing Startle Modulated Prepulse Inhibition with ABR Wave One Amplitudes

It has been shown that suprathreshold prepulse modulation of the startle reflex can be diminished in mice with age-related hearing dysfunctions. Willott et al., Behavioral Neuroscience, 108, 703-713 (1994). This reduction was also demonstrated in the present study with prepulse modulated startle responses and associated inhibition after sound exposure (FIG. 8A1-F1). In agreement with previous studies (Kujawa S G, Liberman M C, J Neurosci 29, 14077-14085 (2009)), ABR amplitudes in exposed mice decreased over time (FIG. 8A2-F2). The most important finding in this study was that the magnitude of these suprathreshold deficits were frequency specific when measured with both PPI audiometry and ABR wave one amplitudes (FIG. 9A, B respectively). Importantly, the greatest deficit was at the upper frequencies or slightly higher than the frequency range of the sound exposure. The damage caused by sound exposure typically localizes at 0.5 to 1 octave above the center frequency range of exposure. Responses to both the low intensity and high intensity sounds were examined using PPI and wave one ABR in an attempt to differentiate the contribution of high and low threshold fibers to hearing loss. The ABR deficits seen at three months were similar spectrally for both 40 and 80 dB SPL, however in agreement with previous work the wave one ABR amplitudes to 80 dB SPL sounds showed the highest extent of changes from control levels (FIG. 9B). Lin et al., J Assoc Res Otolaryngol., 12(5):605-16 (2011). The suprathreshold deficits assessed with PPI and ABR amplitudes were very similar. As mentioned previously, post-exposure PPI functions tend to “flatten” (FIG. 4D; 8A1-F1). This is presumably because the pure tone prepulses are less audible to the animal and thus, less able to modulate the startle stimulus. In unexposed animals high intensity prepulses will suppress the startle almost entirely (FIG. 8A1-F1), however if the animal cannot detect the high intensity prepulses, the startle will not be inhibited, thus creating a flat startle function (FIG. 8A1-F1). This can lead to decreased overall inhibition in response to these suprathreshold stimulus (FIG. 8A2-F2). This data, along with the frequency specific permanent threshold shifts detected by PPI audiometry (FIG. 7A), suggest that this method allows for accurate behavioral measurements of hearing. Furthermore, it is interesting to address that one day after sound exposure, overall startle responses were immediately reduced in both input-output functions (FIG. 10) and in prepulse modulated startle situations (FIG. 8A1-F1). This directly contrasts the change in inhibition which in most cases did not show a significant deficit (at frequencies neighboring sound exposure as seen in FIG. 9A) until three months after exposure. This suggests that ANF carrying broadband startle stimulus information are immediately affected by sound exposure, due to a temporary threshold shift, whereas, frequency specific auditory nerve fiber loss will develop after exposure. This result cannot be detected by ABR wave one amplitudes, as they are not able to be collected one day after exposure due to high temporary threshold shifts.

The PPI Model

Considerations and Limitations of PPI Threshold Assessments

Several important factors must be considered when using the cubic spline function to assess auditory thresholds via PPI functions (FIG. 4). First, while the cubic spline function is a statistically appropriate method to assess large sample sizes at fine resolutions, for reasons mentioned above, it could possibly exaggerate beyond actual behavioral thresholds (Radizwon et al., J. Comp. Physiol. A. Neuroethol. Sens. Neural Behay. Physiol., 195(10), 961-969 (2009)) when the magnitude of the startle response is very small, for example due to hearing loss. Future studies might apply alternatives to the cubic spline function to assess the prepulse modulated startle function. Our mouse data together with a previous study on rats (Fechter et al., J. Acoustic. Soc. Am. 84(1), 179-185 (1988)) advocate that the cubic spline is well-suited for threshold detection in this acoustic startle paradigm. Second, after noise exposure, the absolute startle magnitude is often reduced to at least half of its baseline levels (FIG. 10). This is likely a two part process: first, at one day after exposure the ANF synapse is likely swollen, leading to a significant temporary threshold shift, which is likely to contribute to the reduced startle magnitude. Lobarinas et al., Hearing Research 295, 150-160 (2013). Second, after a period of recovery from temporary threshold shift, the startle reduction seen at the three month time period is likely representing frequency specific detachment of ribbon synapses and concurrent declines of ANFs as a result of intense sound exposure, as evidenced by reduction of wave one ABR amplitudes (FIG. 8A3-F3; FIG. 9B), again contributing to reduced startle magnitude. Kujawa S G, Liberman M C, Hearing Research 330: 191-199 (2015). A drop in startle magnitude after exposure can reduce the dynamic range of the prepulse modulated startle (FIGS. 8A1-F1) and the resulting PPI (FIG. 8A2-F2), especially at three months after exposure when there is presumptive ANF loss. Thus both temporary and permanent changes contribute to a reduced startle amplitude. While the absolute levels of inhibition remain similar to those of unexposed mice at low prepulse intensities (FIGS. 8A2-F2), it is important to remember that this inhibition is the result of a ratio that does not account for changes in dynamic range as a result of cochlear trauma. Psychoacoustically this would imply that inhibition can still occur for low intensity prepulses because the low threshold fibers are intact (FIGS. 8A2-F2). However, due to net startle changes after cochlear loss, inhibition slopes are flatter, which would lead to artificial increases in detection thresholds (FIG. 4D). This is especially true for frequencies most affected by sound exposure (12.5-25 kHz) (FIG. 7A). Thus, it is important to consider changes in the startle reflex magnitude when making conclusions about prepulse inhibition following sound exposure. Lobarinas et al., Research 295, 150-160 (2013).

A Behavioral Representation of High Threshold ANF Depletion Following Sound Exposure

Recent studies have discovered that while clinically normal threshold levels may be maintained following sound exposure, both mice (100 dB SPL 2 hours; Kujawa S G & Liberman M C, J Neurosci 29, 14077-14085 (2009)) and guinea pigs (106 or 109 dB SPL 2 hours; Lin et al., J Assoc Res Otolaryngol., 12(5):605-16 (2011)) display clear suprathreshold ABR wave one amplitude deficits associated with ANF degeneration. Similarly our exposed mice demonstrate reduced ABR wave one amplitudes (FIG. 8A3-F3). Our ABR wave one amplitudes at intense, suprathreshold sound levels were depressed further than at lower sound levels, which might be expected from an intense sound exposure (116 dB SPL 1 hour). The hidden hearing loss model proposed by Schaette and McAlpine suggests that high threshold low spontaneous rate auditory nerve fibers are preferentially damaged after exposure, leaving the majority of low threshold fibers intact (FIG. 12). Schaette R, McAlpine D, J Neurosci 31(38): 13452-13457 (2011). If so, we would expect that the exposed mice in our study would have difficulty perceiving high intensity prepulses at frequencies most affected by sound exposure. These deficits should be mostly evident within a half octave above the center of exposure (in our case 12.5-25 kHz). Cody A R, Johnstone B M, J. Acoust. Soc. Am. 70(3), 707-711 (1981).

Importantly, our PPI methodology was able to test this hypothesis behaviorally. Both the frequency specific ANF damage as well as a general decrease in startle magnitude can be we explained by this model (FIG. 12). The level of inhibition dropped precipitously in response to sound levels above 40 dB SPL at the frequencies most affected by sound exposure (FIG. 8A2-F2). These sound levels are predominantly processed by high threshold auditory nerve fibers. Winter I M, Palmer A R, J. Acoust. Soc. Am. 90(4): 1958-1967 (1991). The loss of these fibers presumably produced the frequency specific prepulse inhibition deficits, a behavioral indication of hidden hearing loss (FIG. 12, high threshold ANF damage). It is also important to highlight the fact that some low threshold fibers were also likely damaged after a 116 dB SPL sound exposure, particularly those fibers responsible for frequencies near 16 kHz (FIG. 12, low threshold ANF damage). This was experimentally observed at 16 kHz with a unique PPI deficit at even at low intensity prepulses (FIG. 8C2), as well as the most elevated detection threshold (FIG. 7A), and the greatest 40 dB SPL stimulus ABR wave one deficit (FIG. 9B). Additionally, this model could also explain why the startle magnitude decreased after exposure (FIG. 8A1-F1, FIG. 10). Since a broadband startle stimulus was used, you would expect the reduced startle response reductions to be proportionate to the high threshold ANF loss across all frequencies showing prepulse inhibition deficits (FIG. 12, high threshold ANF damage).

Can PPI audiometry explain possible perceptual deficits after high intensity sound exposure? Above we discussed how decreases in inhibition might be explained by ANF fiber loss, but it is possible that these deficits have central origin. Interestingly, the inhibition curve is increasingly depressed at higher intensity prepulses (FIG. 8A2-F2), but not closely following the ABR wave one amplitude function (FIG. 8A3-F3). This implies that the behavioral significance of high threshold auditory nerve fibers might be more severe than previously thought. Clearly the direct loss of these fibers alone, which degenerate from the time of exposure until three months after exposure (as evidenced by changes in inhibition from one day after exposure compared to three months in FIG. 8A2-F2), cannot fully explain the intensity coding issues or dynamic range issues that lead to some of the perceptual PPI deficits shown here. Any number of additional plastic changes in the auditory system following ANF degeneration could lead to these extreme inhibition deficits at high intensity prepulses. Even after unilateral sound exposure it is possible that maladaptive plasticity could lead to bilateral changes throughout the auditory system. Popescu M V, Polley D B, Neuron 65, 718-731 (2010). To our knowledge this is one of the first examples of behavioral evidence of hidden hearing loss in an animal model. Mehraei et al., J Neurosci., 36(13): 3755-3764 (2016). Since a modified version of this PPI audiometric method has already been conducted on humans, it might be possible to determine if these effects in mice are also observed in older adults or sound exposed populations. This would be expected based on recent studies showing that ANFs degenerate as a function of age in all humans. Viana et al., Hearing Research 327: 78-88 (2015).

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of detecting hearing loss in a subject, comprising the steps of:
(a) delivering a pre-pulse auditory stimulus to the subject,
(b) allowing a stimulus gap from 10 to 200 milliseconds to pass,
(c) delivering an auditory startle stimulus to the subject,
(d) measuring the startle response of the subject, and determining if the startle response of the subject to the auditory startle stimulus has decreased compared with a control value for the startle response,
repeating steps (a) through (d) until the delivered pre-pulse auditory stimulus has a decibel value sufficient to cause a decrease in the startle response of the subject, and
(e) determining that the subject has hearing loss if the pre-pulse auditory stimulus required to decrease the startle response is higher than a pre-pulse auditory stimulus threshold value for the subject.

2. The method of claim 1, wherein a plurality of subjects are evaluated.

3. The method of claim 2, wherein the plurality of subjects are evaluated simultaneously.

4. The method of claim 1, wherein the subject is a non-human test animal.

5. The method of claim 1, wherein the subject is a human infant.

6. The method of claim 1, wherein the method is repeated using pre-pulse auditory stimulus at a plurality of different audible frequencies.

7. The method of claim 1, wherein the startle stimulus has a loudness of at least 70 decibels sound pressure level.

8. The method of claim 1, wherein a startle algorithm is used to measure the startle response of the subject.

9. The method of claim 1, wherein the method further comprises administering a test drug to the subject before delivering a pre-pulse auditory stimulus to the subject.

10. A method of determining a pre-pulse auditory stimulus threshold value for a subject, comprising the steps of:
(a) delivering a pre-pulse auditory stimulus to the subject,
(b) allowing a stimulus gap from 10 to 200 milliseconds to pass,
(c) delivering an auditory startle stimulus to the subject,
(d) measuring the startle response of the subject, and
(e) identifying the pre-pulse auditory stimulus as a pre-pulse auditory stimulus threshold value for the subject when the measured startle response is lower than a control value for a startle response.

11. The method of claim 10, wherein the method is used to determine a plurality of pre-pulse auditory stimulus threshold values for a plurality of subjects.

12. The method of claim 11, wherein the plurality of pre-pulse auditory stimulus threshold values are determined simultaneously.

13. The method of claim 10, wherein the subject is a non-human test animal.

14. The method of claim 10, wherein the subject is a human infant.

15. The method of claim 10, wherein the method is repeated using pre-pulse auditory stimulus at a plurality of different audible frequencies.

16. The method of claim 10, wherein the startle stimulus has a loudness of at least 70 decibels sound pressure level.

17. A method of determining the effect of a drug on the hearing of a subject,
(a) determining a pre-pulse auditory stimulus threshold value for the subject, comprising the steps of:
(1) delivering a pre-pulse auditory stimulus to the subject,
(2) allowing a stimulus gap from 10 to 200 milliseconds to pass,
(3) delivering an auditory startle stimulus to the subject,
(4) measuring the startle response of the subject, and
(5) identifying the pre-pulse auditory stimulus as a pre-pulse auditory stimulus threshold value for the subject when the measured startle response is lower than a control value for a startle response.
(b) administering a drug to the subject, and
(c) determining the effect of the drug on hearing of the subject, comprising the steps of:
(1) delivering a pre-pulse auditory stimulus to the subject,
(2) allowing a stimulus gap from 10 to 200 milliseconds to pass,
(3) delivering an auditory startle stimulus to the subject,
(4) measuring the startle response of the subject, and determining if the startle response of the subject to the auditory startle stimulus has decreased compared with a control value for the startle response,
repeating steps c(1) through c(4) until the delivered pre-pulse auditory stimulus has a value sufficient to cause a decrease in the startle response of the subject, and
(5) determining that the drug has caused hearing loss in the subject if the pre-pulse auditory stimulus required to decrease the startle response is higher than the pre-pulse auditory stimulus threshold value for the subject or determining that the drug has caused hearing improvement in the subject if the pre-pulse auditory stimulus required to decrease the startle response is lower than the pre-pulse auditory stimulus threshold value for the subject.

18. The method of claim 17, wherein the method is used to determine a plurality of pre-pulse auditory stimulus threshold values for a plurality of subjects.

19. The method of claim 17, wherein the subject is a non-human test animal.

20. The method of claim 17, wherein the method is repeated using pre-pulse auditory stimulus at a plurality of different audible frequencies.
